(19) United States
(12) Patent Application Publication
(10) Pub. No.: US 2008/0159980 A1
(43) Pub. Date: Jul. 3, 2008

(54) Combination cancer therapy with a GST-activated anticancer compound and another anticancer therapy

(75) Inventors: Hua Xu, Sunnyvale, CA (US); Gail L. Brown, Woodside, CA (US); Steven R. Schow, Redwood City, CA (US); James G. Keck, Redwood City, CA (US)

Correspondence Address: FOLEY & LARDNER LLP, 975 PAGE MILL ROAD, PALO ALTO, CA, 94304, US

(73) Assignee: Telik, Inc.

(21) Appl. No.: 11/872,659

(22) Filed: Oct. 15, 2007

Related U.S. Application Data
(63) Continuation of application No. 10/714,593, filed on Nov. 14, 2003, now abandoned.
(60) Provisional application No. 60/426,983, filed on Nov. 15, 2002.

(51) Int. Cl. A61K 31/664; A61K 38/20; A61K 38/21; A61K 39/395; A61K 31/704; A61P 35/00

(52) U.S. Cl. 424/85.2; 514/118; 424/85.4; 424/141.1; 514/34

(57) ABSTRACT

A method of combination cancer therapy in a mammal, especially a human, by administering a therapeutically effective amount of a GST-activated anticancer compound and a therapeutically effective amount of another anticancer therapy, that is, an anticancer therapy that is not a treatment with a GST- activated anticancer compound (including chemotherapy; molecular targeted therapy; biologic therapy; and radiotherapy, used as monotherapy or in combination). Pharmaceutical compositions, products, and kits for the method. The use of a GST-activated anticancer compound in the manufacture of a medicament for the method. A method of potentiating an anticancer therapy in a mammal, especially a human, comprising administering a therapeutically effective amount of a GST-activated anticancer compound to the mammal being treated with the anticancer therapy. The use of a GST-activated anticancer compound in the manufacture of a medicament for the method. The GST-activated anticancer compound is preferably a compound of U.S. Pat. No. 5,556,942, and more preferably TLK286, especially as the hydrochloride salt.

### CROSS-REFERENCE TO RELATED APPLICATIONS

This application is a continuation and claims the benefit under 35 U.S.C. 120 of U.S. application Ser. No. 10/714,593, filed 14 Nov. 2003, which in turn claims the benefit under 35 USC 119(e) of U.S. Provisional Application No. 60/426,983, filed 15 Nov. 2002. The entire contents of both of these prior applications are incorporated into this application by reference.

### BACKGROUND OF THE INVENTION

1. Field of the Invention

This invention relates to cancer therapy.

2. Description of the Related Art

The purpose of cancer therapy (anticancer therapy) is to prevent cancer cells from multiplying, invading, metastasizing, and ultimately killing their host organism, e.g. a human or other mammal. Because cell multiplication is a characteristic of many normal cells as well as cancer cells, most anticancer therapies also have toxic effects on normal cells, particularly those with a rapid rate of turnover, such as bone marrow and mucous membrane cells. The goal in selecting an effective cancer therapy, therefore, is to find a therapy that has a marked growth inhibitory or controlling effect on the cancer cells and a minimal toxic effect on the host. In the most effective therapies, the agents used are capable not only of inhibiting but also eradicating all cancer cells while sufficiently preserving normal cells to permit the host to return to normal or at least satisfactory life function and quality. Cancer therapies include classic chemotherapy with antiproliferative agents (typically, small molecules) that target all dividing cells; molecular targeted therapy designed to specifically target cancer cells, such as functional therapy designed to alter a molecular function in the cancer cells with gene therapy, antisense therapy, and drugs such as erlotinib hydrochloride, gefitinib, and imatinib mesylate, and phenotype-directed therapy designed to target the unique phenotype of cancer cells such as therapy with monoclonal antibodies, immunotoxins, radioimmunoconjugates, and cancer vaccines; biologic therapy with cytokines such as interleukin-2 and interferon-α; and radiotherapy.

However, although the first effective anticancer compounds were brought into clinical trials in the 1940′s, initial therapeutic results were disappointing. Regressions of acute lymphocytic leukemia and adult lymphomas were obtained with single agents such as the nitrogen mustards, antifolates, corticosteroids, and vinca alkaloids, but responses were frequently partial and only of short duration; and relapse was associated with resistance to the original drug. Initial resistance to a given single agent (natural resistance) is frequent, and even initially responsive cancers frequently display acquired resistance after drug exposure, probably owing to selection of pre-existing resistant cancer cells from a heterogeneous population and possibly also owing to an increased rate of mutation to resistance. This is consistent with the clinical observation that, with few exceptions, cancers are cured only by combination therapy. Cancers are frequently characterized as being resistant (not showing a response during the initial course of therapy) or refractory (having shown an initial response, then relapsed, and not showing a response on a later course of therapy) to anticancer therapies. Resistance to one anticancer drug, e.g. a platinum anticancer compound such as cisplatin, is often associated with cross-resistance to other drugs of the same class, e.g. other platinum compounds. Multiple drug resistance, also called pleiotropic drug resistance, is a phenomenon where treatment with one drug confers resistance not only to that drug and others of its class but also to unrelated agents.

Anticancer therapies, especially chemotherapies, are frequently employed in combination, for several principal reasons. First, treatment with two or more non-cross-resistant therapies may prevent the formation of resistant clones; second, the combination of two or more therapies that are active against cells in different phases of growth (resting—G₀, postmitotic—G₀, DNA synthesis—S, premitotic—G₂, and mitotic—M) may kill cells that are dividing slowly as well as those that are dividing actively and/or recruit cells into a more actively dividing state, making them more sensitive to many anticancer therapies; and third, the combination may create a biochemical enhancement effect by affecting different pathways or different steps in a single biochemical pathway. Particularly when the toxicities of the therapies are non-overlapping, two or more therapies may be employed in full or nearly full amounts, and the effectiveness of each therapy will be maintained in the combination; thus, traditional myelosuppressive drugs may be supplemented by non-myelosuppressive drugs such as the vinca alkaloids, prednisone, and bleomycin; and combination chemotherapies have been developed for a number of cancers that are not curable with single agents. Combinations of two or more of chemotherapy, molecular targeted therapy, biologic therapy, and radiotherapy are also known and used. Although the existence of a wide variety of mechanistically distinct anticancer therapies suggests that non-cross-resistant therapies can be found, cancer cells are known to possess a variety of mechanisms that confer pleiotropic drug resistance. These mechanisms of resistance contribute to the failure of combination therapy to cure common cancers such as metastatic colon cancer and prostate cancer.

Discussions of anticancer chemotherapy and biologic therapy, and examples of suitable therapeutic protocols, may be found in such books as Cancer Chemotherapy and Biotherapy: Principles and Practice, 3rd ed. (2001), Chabner and Longo, eds., and Handbook of Cancer Chemotherapy, 6th ed. (2003), Skeet, ed., both from Lippincott Williams & Wilkins, Philadelphia, Pa., U.S.A.; and regimens for anticancer therapies, especially chemotherapies, may be found on Web sites such as those maintained by the National Cancer Institute (www.cancer.gov), the American Society for Clinical Oncology (www.asco.org), and the National Comprehensive Cancer Network (www.nccn.org).

Glutathione (GSH), in its reduced form, is a tripeptide of the formula: γ-L-Glu-L-Cys-Gly. Reduced glutathione has a central role in maintaining the redox condition in cells and is also an essential substrate for glutathione S-transferase (GST). GST exists in mammals as a superfamily of isoenzymes which regulate the metabolism and detoxification of foreign substances introduced into cells. In general, GST can facilitate detoxification of foreign substances (including anticancer drugs), but it can also convert certain precursors into toxic substances. The isoenzyme GST P1-1 is constitutively expressed in many cancer cells, such as ovarian, non-small cell lung, breast, colorectal, pancreatic, and lymphoma tissue (more than 75% of human tumor specimens from breast, lung, liver, and colorectal cancers are reported to express GST P1-1). It is frequently overexpressed in tumors following treatment with many chemotherapeutic agents, and is seen in cancer cells that have developed resistance to these agents.

U.S. Pat. No. 5,556,942 discloses compounds of the formula

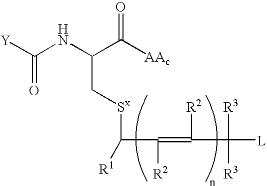

and their amides, esters, and salts, where:
L is an electron withdrawing leaving group;
S^{x} is —S(═O)—, —S(═O)₂, —S(═NH)—, —S(═O)(═NH)—, —S+(C₁-C₆ alkyl)-, —Se(═O)—, —Se(═O)₂—, —Se(═NH)—,

### or —Se(═O)(═NH)—, or is —O—C(═O)—, or —HN—C(═O)—;

each R¹, R² and R³ is independently H or a non-interfering substituent;
n is 0, 1 or 2;
Y is selected from the group consisting of

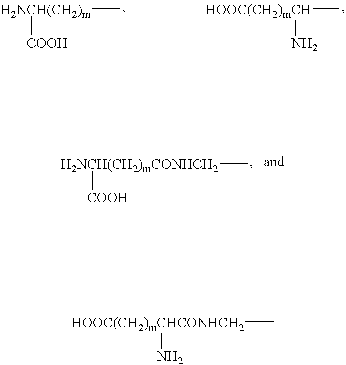

where m is 1 or 2; and
AA_{c} is an amino acid linked through a peptide bond to the remainder of the compound, and their syntheses.

The compounds of the patent are stated to be useful drugs for the selective treatment of target tissues which contain compatible GST isoenzymes, and simultaneously elevate the levels of GM progenitor cells in bone marrow. Disclosed embodiments for L include those that generate a drug that is cytotoxic to unwanted cells, including the phosphoramidate and phosphorodiamidate mustards.

TLK286, identified in the patent as TER 286 and named as γ-glutamyl-α-amino-β-((2-ethyl-N,N,N,N-tetra(2′-chloro) ethylphosphoramidate) sulfonyl)propionyl-(R)-(-)phenylglycine, is one of these compounds. TLK286 is the compound of the formula

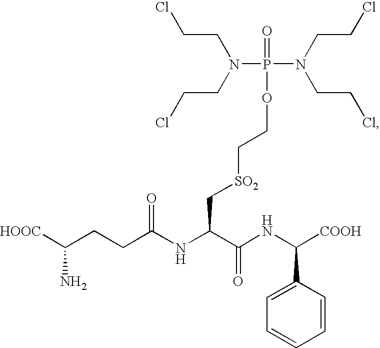

and has the CAS name L-γ-glutamyl-3-[[2-[[bis[bis (2-chloroethyl)amino]phosphinyl]oxy]ethyl]sulfonyl]-L-alanyl-2-phenyl-(2R)-glycine. TLK286 as the hydrochloride salt has the United States Adopted Name of canfosfamide hydrochloride. TLK286 is an anticancer compound that is activated by the actions of GST P1-1, and by GST A1-1, to release the cytotoxic phosphorodiamidate mustard moiety. Following activation of TLK286 by GST P1-1, apoptosis is induced through the stress response signaling pathway with the activation of MKK4, JNK, p38 MAP kinase, and caspase 3.

In vitro, TLK286 has been shown to be more potent in the M6709 human colon carcinoma cell line selected for resistance to doxorubicin and the MCF-7 human breast carcinoma cell line selected for resistance to cyclophosphamide, both of which overexpress GST P1-1, over their parental cell lines; and in murine xenografts of M7609 engineered to have high, medium, and low levels of GST P1-1, the potency of TLK286 was positively correlated with the level of GST P1-1 (Morgan et al., Cancer Res., 58:2568 (1998)).

TLK286, as its hydrochloride salt, is currently being evaluated in multiple clinical trials for the treatment of ovarian, breast, non-small cell lung, and colorectal cancers. It has demonstrated significant single agent antitumor activity and improvement in survival in patients with non-small cell lung cancer and ovarian cancer, and single agent antitumor activity in colorectal and breast cancer. Evidence from in vitro cell culture and tumor biopsies indicates that TLK286 is non-cross-resistant to platinum, paclitaxel, and doxorubicin (Rosario et al., Mol. Pharmacol., 58:167 (2000)), and also to gemcitabine. Patients treated with TLK286 show a very low incidence of clinically significant hematological toxicity.

Other compounds specifically mentioned within U.S. Pat. No. 5,556,942 are TLK231 (TER 231), L-γ-glutamyl-3-[[2-[[bis[bis(2-chloroethyl)amino]phosphinyl]oxy]ethyl]sulfonyl]-L-alanyl-glycine, activated by GST M1a-1a; TLK303 (TER 303), L-γ-glutamyl-3-[[2-[[bis[bis(2-choroethyl)-amino]phosphinyl]oxy]ethyl]sulfonyl]-L-alanyl-2-phenyl-(2S) -alanine, activated by GST A1-1; TLK296 (TER 296), L-γ-glutamyl-3-[[2-[[bis[bis(2-chloroethyl)amino]phosphinyl]oxy]ethyl]sulfonyl]-L-phenylalanyl-glycine, activated by GST P1-1; and TLK297 (TER 297), L-γ-glutamyl-3-[[2-[[bis[bis(2-chloroethyl)amino]phosphinyl]oxy]ethyl]sulfonyl]-L-phenylalanyl-2-phenyl-(2R) -glycine, and their salts.

The disclosure of U.S. Pat. No. 5,556,942, and the disclosures of other documents referred to in this application, are incorporated into this application by reference.

Cancer therapies are steadily evolving, but it remains true that even the best current therapies are not always even initially effective and frequently become ineffective after treatment, so that improved cancer therapies are constantly being sought.

### SUMMARY OF THE INVENTION

In a first aspect, this invention is a method of combination cancer therapy in a mammal, especially a human, comprising administering a therapeutically effective amount of a GST-activated anticancer compound and a therapeutically effective amount of another anticancer therapy, that is, an anticancer therapy that is not a treatment with a GST-activated anticancer compound (including chemotherapy; molecular targeted therapy; biologic therapy; and radiotherapy, used as monotherapy or in combination).

In a second aspect, this invention is a method of potentiating an anticancer therapy in a mammal, especially a human, comprising administering a therapeutically effective amount of a GST-activated anticancer compound to the mammal being treated with the anticancer therapy.

In a third aspect, this invention is a pharmaceutical composition for anticancer therapy comprising a GST-activated anticancer compound, one or more of another anticancer chemotherapy agent, a molecular targeted therapy agent, or a biologic therapy agent, and an excipient.

In a fourth aspect, this invention is a pharmaceutical product or kit for anticancer therapy comprising a GST-activated anticancer compound in dosage form and one or more of another anticancer chemotherapy agent, a molecular targeted therapy agent, or a biologic therapy agent, also in dosage form.

In a fifth aspect, this invention is the use of a GST-activated anticancer compound and one or more of another anticancer chemotherapy agent, a molecular targeted therapy agent, or a biologic therapy agent, in the manufacture of a medicament for the treatment of cancer in a mammal, especially a human.

In a sixth aspect, this invention is the use of a GST-activated anticancer compound in the manufacture of a medicament for the treatment of cancer in a mammal, especially a human, that is being treated with radiation therapy.

In preferred embodiments of this invention (preferred embodiments of the methods, compositions, products, kits, and uses of this invention as mentioned in paragraphs [0016] through [0021] above), the GST-activated anticancer compound is a compound of U.S. Pat. No. 5,556,942, especially TLK286 or an amide, ester, amide/ester, or salt thereof, particularly TLK286 or a salt thereof, especially TLK286 hydrochloride; and these preferences and preferred another anticancer therapies with which the therapy with the GST-activated anticancer compound may be combined are characterized by the specification and by the features of method claims 2 through 20 of this application as filed.

In a particular embodiment of the invention, the combination cancer therapy of this invention excludes combination therapy with the two-drug combination of TLK286 and docetaxel; or includes combination therapy with the two-drug combination TLK286 and docetaxel only with dosages of TLK286 of 60-1280 mg/m², especially 400-1000 mg/M², and dosages of docetaxel of 35-100 mg/M², especially 50-100 mg/m².

### BRIEF DESCRIPTION OF THE DRAWINGS

FIG. 1 shows the inhibition of growth of OVCAR-3 cells treated with carboplatin, TLK286, and carboplatin+TLK286.

FIG. 2 shows the inhibition of growth of DLD-1 cells treated with oxaliplatin, TLK286, and oxaliplatin+TLK286.

FIG. 3 shows the inhibition of growth of OVCAR-3 cells treated with doxorubicin, TLK286, and doxorubicin+TLK286.

FIG. 4 shows the inhibition of proliferation of MCF-7 cells treated with docetaxel, TLK286, and docetaxel +TLK286.

FIG. 5 shows the inhibition of proliferation of A-549 cells treated with cisplatin, TLK286, and cisplatin +TLK286.

FIG. 6 shows the inhibition of proliferation of A-549 cells treated with paclitaxel, TLK286, and paclitaxel +TLK286.

FIG. 7 shows the inhibition of growth of MCF-7 cells treated with gemcitabine, TLK286, and gemcitabine +TLK286.

FIG. 8 shows the inhibition of growth of RL cells treated with rituximab, TLK286, and rituximab +TLK286.

FIG. 9 shows the inhibition of growth of MX-1 cells treated with gefitinib, TLK286, and gefitinib +TLK286.

### DETAILED DESCRIPTION OF THE INVENTION

The GST-activated anticancer compound

A “GST-activated anticancer compound” is a compound comprising glutathione or a glutathione analog chemically linked to a cytotoxic moiety such that the cytotoxic moiety is released by cleavage from the glutathione or glutathione analog in the presence of one or more GST isoenzymes.

Suitable such compounds include those disclosed in U.S. Pat. No. 5,556,942 and are of the formula

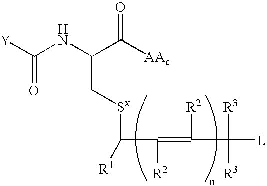

and their amides, esters, and salts, where:
L is a cytotoxic electron withdrawing leaving group;
S^{x} is —S(═O)—, —S(═O)₂, —S(═NH)—, —S(═O)(═NH)—, —S+(C₁-C₆ alkyl)-, —Se(═O)—, —Se(═O)₂—, —Se(═NH)—,

### or —Se(═O)(═NH)—, or is —O—C(═O)—, or —HN—C(═O)—;

each of R¹, R² and R³ is independently H or a non-interfering substituent, such as H, optionally substituted C₁-C₆ alkyl (for example, methyl, tert-butyl, cyclohexyl, and the like), optionally substituted C₆-C₁₂ aryl (for example, phenyl, naphthyl, pyridyl, and the like), optionally substituted C₇-Cl₂ aralkyl (for example, benzyl, phenylethyl, 2-pyridylethyl, and the like), cyano, halo, optionally substituted C₇-C₆ alkoxy, optionally substituted C₆-C₁₂ aryloxy, or optionally substituted C₇-C₁₂ aralkoxy, where the substituents may be halo, —OR, —SR; and —NR₂, where R is H or C₁-C₄ alkyl;
n is 0, 1 or 2;
Y is selected from the group consisting of

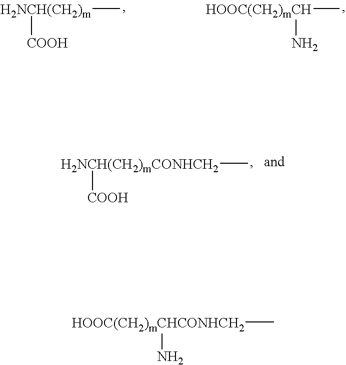

where m is 1 or 2; and

AA_{c} is an amino acid linked through a peptide bond to the remainder of the compound.

In preferred embodiments, one or more of the following preferences is met:

L is a toxin such as ricin or diphtheria toxin, a linkable anticancer agent such as doxorubicin or daunorubicin, or a phosphoramidate or phosphorodiamidate mustard, especially a phosphorodiamidate mustard of the formula —OP(═O)(NHCH₂CH₂X)₂ or —OP(═O)(N(CH₂CH₂X)₂)₂, particularly of the formula —OP(═O)(N(CH₂CH₂X)₂)₂, where X is Cl or Br, especially Cl;

### Sₓ is O═S═O;

R¹ is H, C₁-C₄ alkyl, or phenyl, especially H or phenyl, particularly H;
each R² is independently chosen from H and C₁-C₆ alkyl, especially H;
each R³ is independently chosen from H, C₁-C₄ alkyl, and phenyl, especially H;
n is 0;
Y—C(═O)— is γ-glutamyl, β-aspartyl, glutamyl, aspartyl, β-glutamylglycyl, β-aspartylglycyl, glutamylglycyl, or aspartylglycyl, especially γ-glutamyl;
AA_{c} is glycine, phenylglycine, β-alanine, alanine, phenylalanine, valine, 4-aminobutyric acid, aspartic acid, histidine, tryptophan, and tyrosine, as either the (S)- or (R)-isomers, optionally substituted on the phenyl ring as described above for R¹ through R³, especially glycine, phenylglycine, β-alanine, alanine, or phenylalanine, and particularly (R)-phenylglycine.

Suitable amides and esters of these compounds include those in which one or more of the carboxyl groups is amidated or esterified to form a C₁-C₆ alkyl or alkenyl, C₆-C₁₀ aryl, or C₇-C₁₂ aralkyl amide or ester, in which the alkyl or aryl groups may be optionally substituted with noninterfering substituents such as halo, alkoxy, or alkylamino. The amides and esters may be monoamides, diamides, or (if applicable) triamides, monoesters, diesters, or (if applicable) triesters, or mixed amide-esters. Suitable salts (see Berge et al., J. Pharm. Sci., 66:1 (1971) for a nonexclusive list) are those formed when inorganic bases (e.g. sodium, potassium, and calcium hydroxide) or organic bases (e.g. ethanolamine, diethanolamine, triethanolamine, ethylenediamine, tromethamine, N-methylglucaamine) react with the carboxyl groups, and those formed when inorganic acids (e. g hydrochloric, hydrobromic, sulfuric, nitric, and chlorosulfonic acids) or organic acids (e.g. acetic, propionic, oxalic, malic, maleic, malonic, fumaric, or tartaric acids, and alkane- or arenesulfonic acids such as methanesulfonic, ethanesulfonic, benzenesulfonic, substituted benzenesulfonic such as chlorobenzenesulfonic and toluenesulfonic, naphthalenesulfonic and substituted naphthalenesulfonic, naphthalenedisulfonic and substituted naphthalenedisulfonic, and camphorsulfonic acids) react to form acid addition salts of the amine groups. Mixed amide salts and ester salts are also included, as are hydrates and other solvates as well as unsolvated forms.

The preparation of these compounds and their derivatives may be made by methods well known to the person of ordinary skill in the art and as described in U.S. Pat. No. 5,556,942.

A particularly preferred GST-activated anticancer compound is TLK286, as its hydrochloride salt (throughout the specification, reference to TLK286 should be taken to mean TLK286 as its hydrochloride salt).

As a monotherapy for a number of cancers, including ovarian, breast, non-small cell lung, and colorectal cancers, TLK286 has been administered by intravenous infusion at doses of 400-1000 mg/m² body surface area at once/week and once/three weeks.

As a combination therapy with docetaxel (75 mg/m²), TLK 286 has been administered at 500, 750, and 960 mg/m² at 3-weekly intervals. As a combination therapy with carboplatin (AUC 5 or 6 mg/mL-·min), TLK 286 has been administered at 500, 750, and 960 mg/m² at 3- to 4-weekly intervals. As a combination therapy with liposomal doxorubicin (40 or 50 mg/m²), TLK 286 has been administered at 500, 750, and 960 mg/m² at 4-weekly intervals.

Another Anticancer Therapy

“Another anticancer therapy” is an anticancer therapy that is not a treatment with a GST-activated anticancer compound, especially a compound disclosed in paragraphs [0034] to [0037] above. Such “another anticancer therapies” include classic chemotherapy; molecular targeted therapy; biologic therapy; and radiotherapy. These therapies are those used as monotherapy or in combination therapy.

Chemotherapeutic Agents Include:

alkylating agents, including:
alkyl sulfonates such as busulfan,
ethyleneimine derivatives such as thiotepa,
nitrogen mustards such as chlorambucil, cyclophosphamide, estramustine, ifosfamide, mechlorethamine, melphalan, and uramustine,
nitrosoureas such as carmustine, lomustine, and streptozocin,
triazenes such as dacarbazine, procarbazine, and temozolamide, and
platinum compounds such as cisplatin, carboplatin, oxaliplatin, satraplatin, and (SP-4-3)-(cis)-amminedichloro-[2-methylpyridine]platinum(II);
antimetabolites, including:
antifolates such as methotrexate, permetrexed, raltitrexed, and trimetrexate,
purine analogs such as cladribine, chlorodeoxyadenosine, clofarabine, fludarabine, mercaptopurine, pentostatin, and thioguanine,
pyrimidine analogs such as azacitidine, capecitabine, cytarabine, edatrexate, floxuridine, fluorouracil, gemcitabine, and troxacitabine;
natural products, including:
antitumor antibiotics such as bleomycin, dactinomycin, mithramycin, mitomycin, mitoxantrone,
porfiromycin, and anthracyclines such as daunorubicin (including liposomal daunorubicin), doxorubicin (including liposomal doxorubicin), epirubicin, idarubicin, and valrubicin,
enzymes such as L-asparaginase and PEG-L-asparaginase,
microtubule polymer stabilizers such as the taxanes paclitaxel and docetaxel,
mitotic inhibitors such as the vinca alkaloids vinblastine, vincristine, vindesine, and vinorelbine,
topisomerase I inhibitors such as the camptothecins irinotecan and topotecan, and
topoisomerase II inhibitors such as amsacrine, etoposide, and teniposide;
hormones and hormone antagonists, including:
androgens such as fluoxymesterone and testolactone,
antiandrogens such as bicalutamide, cyproterone, flutamide, and nilutamide,
aromatase inhibitors such as aminoglutethimide, anastrozole, exemestane, formestane, and letrozole,
corticosteroids such as dexamethasone and prednisone,
estrogens such as diethylstilbestrol,
antiestrogens such as fulvestrant, raloxifene, tamoxifen, and toremifine,
LHRH agonists and antagonists such as buserelin, goserelin, leuprolide, and triptorelin,
progestins such as medroxyprogesterone acetate and megestrol acetate, and
thyroid hormones such as levothyroxine and liothyronine; and
miscellaneous agents, including altretamine, arsenic trioxide, gallium nitrate, hydroxyurea, levamisole,
mitotane, octreotide, procarbazine, suramin, thalidomide, photodynamic compounds such as methoxsalen and sodium porfimer, and proteasome inhibitors such as bortezomib.

Molecular Targeted Therapy Agents Include:

functional therapeutic agents, including:
gene therapy agents,
antisense therapy agents,
tyrosine kinase inhibitors such as erlotinib hydrochloride, gefitinib, imatinib mesylate, and semaxanib, and
gene expression modulators such as the retinoids and rexinoids, e.g. adapalene, bexarotene, trans-retinoic acid, 9-cis-retinoic acid, and N-(4-hydroxyphenyl)retinamide;
phenotype-directed therapy agents, including:
monoclonal antibodies such as alemtuzumab, bevacizumab, cetuximab, ibritumomab tiuxetan, rituximab, and trastuzumab,
immunotoxins such as gemtuzumab ozogamicin,
radioimmunoconjugates such as ¹³¹-tositumomab, and cancer vaccines.

Biologic therapy agents include:

interferons such as interferon-α₂ₐ and interferon-α_{2b}, and interleukins such as aldesleukin, denileukin diftitox, and oprelvekin.

In addition to these agents intended to act against cancer cells, cancer therapies include the use of protective or adjunctive agents, including:

cytoprotective agents such as armifostine, dexrazonxane, and mesna,
phosphonates such as parmidronate and zoledronic acid, and
stimulating factors such as epoetin, darbeopetin, filgrastim, PEG-filgrastim, and sargramostim.

Combination cancer therapy regimens with which the GST-activated anticancer compound may be combined include all regimens involving the use of two or more of the anticancer therapies (anticancer agents) such as those mentioned in paragraphs [0044] to [0047] above and/or radiotherapy, optionally including protective and adjunctive agents such as those mentioned in paragraph [0048] above; and TLK286 can be added to existing anticancer regimens known for the treatment of various cancers, such as the regimens mentioned in paragraph [0006] above.

Many combination chemotherapeutic regimens are known to the art, such as combinations of platinum compounds and taxanes, e.g. carboplatin/paclitaxel, capecitabine/docetaxel, the “Cooper regimen”, fluorouracil-levamisole, fluorouracil-leucovorin, methotrexate-leucovorin, and those known by the acronyms ABDIC, ABVD, AC, ADIC, AI, BACOD, BACOP, BVCPP, CABO, CAD, CAE, CAF, CAP, CD, CEC, CF, CHOP, CHOP+rituximab, CIC, CMF, CMFP, CyADIC, CyVADIC, DAC, DVD, FAC, FAC-S, FAM-S, FOLFOX-4, FOLFOX-6, M-BACOD, MACOB-B, MAID, MOPP, MVAC, PCV, T-5, VAC, VAD, VAPA, VAP-Cyclo, VAP-II, VBM, VBMCP, VIP, VP, and the like.

Combinations of chemotherapies and molecular targeted therapies, biologic therapies, and radiation therapies are also well known to the art; including therapies such as trastuzumab+paclitaxel, alone or in further combination with carboplatin, for certain breast cancers, and many other such regimens for other cancers; and the “Dublin regimen” (555 mg/m² fluorouracil IV over 16 hours on days 1-5 and 75 mg/m² cisplatin IV over 8 hours on day 7, with repetition at 6 weeks, in combination with 40 Gy radiotherapy in 15 fractions over the first 3 weeks) and the “Michigan regimen” (fluorouracil+cisplatin+vinblastine+radiotherapy), both for esophageal cancer, and many other such regimens for other cancers.

Combination Treatment with a GST-Activated Anticancer Compound and Another Anticancer Therapy

This invention is a method of combination cancer therapy in a mammal, especially a human, by administering a therapeutically effective amount of a GST-activated anticancer compound and a therapeutically effective amount of another anticancer therapy.

“Combination therapy” means the administration of the GST-activated anticancer compound and the another anticancer therapy during the course of cancer chemotherapy. Such combination therapy may involve the administration of the GST-activated anticancer compound before, during, and/or after the administration of the another anticancer therapy. The administration of the GST-activated anticancer compound may be separated in time from the administration of the another anticancer therapy by up to several weeks, and may precede it or follow it, but more commonly the administration of the GST-activated anticancer compound will accompany at least one aspect of the another anticancer therapy (such as the administration of one dose of a chemotherapeutic agent, molecular targeted therapy agent, biologic therapy agent, or radiation therapy) within up to 48 hours, and most commonly within less than 24 hours.

A “therapeutically effective amount” means that amount which, when administered to a mammal, especially a human, for treating a cancer, is sufficient to effect treatment for the cancer.

“Treating” or “treatment” of a cancer in a mammal includes one or more of:
(1) inhibiting growth of the cancer, i.e., arresting its development,
(2) preventing spread of the cancer, i.e. preventing metastases,
(3) relieving the cancer, i.e., causing regression of the cancer,
(4) preventing recurrence of the cancer, and
(5) palliating symptoms of the cancer.

Cancers which may be effectively treated by the method of this invention include mammalian cancers, especially human cancers. Cancers that are particularly treatable by the method of this invention are cancers with sensitivity to inducers of apoptosis, and more specifically those cancers that express or, particularly, overexpress one or more glutathione S-transferase isoenzymes. Cancers that express or overexpress one or more glutathione S-transferase isoenzymes when treated with other anticancer compounds or combination cancer chemotherapy regimens (i.e. those not including a GST-activated anticancer compound) are especially treatable by the method of this invention. Such cancers include cancers of the brain, breast, bladder, cervix, colon and rectum, esophagus, head and neck, kidney, lung, liver, ovary, pancreas, prostate, and stomach; leukemias such as ALL, AML, AMML, CLL, CML, CMML, and hairy cell leukemia; Hodgkin's and non-Hodgkin's lymphomas; mesotheliomas, multiple myeloma; and sarcomas of bone and soft tissue. Cancers particularly treatable by the method of this invention with TLK286 as the GST-activated anticancer compound include breast, ovarian, colorectal, and non-small cell lung cancers; and TLK296 would also be useful for the same cancers because it also is activated by GST P1-1. Other GST-activated anticancer compounds are expected to be suitable for these or other cancers depending on the nature of the GST isoenzymes expressed by the cancer being treated.

The method of this invention comprises combining the administration of a therapeutically effective amount of a GST-activated anticancer compound and a therapeutically active amount of another anticancer therapy. The another anticancer therapy will generally be one that has utility in the treatment of the cancer being treated even without the concomitant administration of the GST-activated anticancer compound; and a suitable such another anticancer therapy for a particular cancer to be treated will be determinable by a person of ordinary skill in the art having regard to that knowledge and this disclosure. It is of course contemplated that the combination therapy of this invention may be used with anticancer therapies not yet in use. The GST-activated anticancer agent may also be used as adjuvant or neoadjuvant therapy accompanying radiation therapy.

The amount of the GST-activated anticancer compound that is administered to the mammal should be a therapeutically effective amount when used in conjunction with the another anticancer therapy; and similarly the amount of the another anticancer therapy that is administered to the mammal should be a therapeutically effective amount when used in conjunction with the GST-activated anticancer compound. However, the therapeutically effective amount of either the GST-activated anticancer compound and the amount of the another anticancer therapy when administered in the combination cancer chemotherapy of this invention may each be less than the amount which would be therapeutically effective if delivered to the mammal alone. It is common in cancer therapy, though, to use the maximum tolerated dose of the or each therapy, with a reduction only because of common toxicity of the therapies used or potentiation of the toxicity of one therapy by another. Because of the lack of cross-resistance of TLK286, for example, with several common chemotherapeutic agents, and its relative lack of clinically severe toxicity, especially its lack of clinically severe hematological toxicity, it is expected that TLK286 will be administrable at essentially its maximum tolerated dose as a single agent, and no reduction in the amount of the another anticancer therapy will be required. Examples 10 through 12 illustrate that this has been shown for three common anticancer agents.

Although not wishing to be bound by theory, it is considered that combination therapy with the GST-activated anticancer compound, particularly a GST P1-1 activated anticancer compound such as TLK286, and another anticancer therapy will be of benefit because of one or both of the following mechanisms:

(1) GST P1-1 is overexpressed when cancer cell lines are treated with known anticancer therapies such as treatment with platinum-containing compounds and doxorubicin; and the rise in GST P1-1 is correlated with an increase in resistance to the anticancer therapy. Because compounds such as TLK286 are activated by GST P1-1 to release the cytotoxic phosphorodiamidate moiety, cancer cells that have been treated with another anticancer therapy will contain an elevated level of GST P1-1 and will therefore increase the activity of TLK286 in these cells, increasing its cytotoxicity. Thus administration of combination therapy with a GST-activated anticancer compound such as TLK286 and another anticancer therapy will make the combination more effective than either therapy alone; and
(2) Compounds such as TLK286 are activated by GST P1-1, and this activation is achieved by interaction of the TLK286 with the active site of the enzyme. This interaction will limit the ability of the enzyme to interact with and detoxify other anticancer agents which might otherwise be detoxified by GST P1-1, thereby effectively increasing the cytotoxicity of these other anticancer agents. Thus administration of combination therapy with a GST-activated anticancer compound such as TLK286 and another anticancer therapy will make the combination more effective than either therapy alone. The additive to synergistic effect of TLK286 with other anticancer therapies is illustrated in the Examples later in the application.

Suitable dosing for TLK286 as the GST-activated anticancer compound is about 60-1280 mg/m² body surface area, especially 500-1000 mg/m². Dosing may be at 1-35 day intervals; for example, about 500-1000 mg/m² at 1-5 week intervals, especially at 1, 2, 3, or 4 week intervals, or at higher frequencies including as frequently as once/day for several (e.g. 5 or 7) days, with the dosing repeated every 2, 3, or 4 weeks, or constant infusion for a period of 6-72 hours, also with the dosing repeated every 2, 3, or 4 weeks; and such dosing flexibility will readily enable combination therapy with the anticancer therapies now used. Suitable dosages and dose frequencies for other GST-activated anticancer compounds will be readily determinable by a person of ordinary skill in the art having regard to that skill and this disclosure.

Suitable dosing for the other anticancer therapy will be the dosing already established for that therapy, as described in such documents as those listed in paragraph [0006]. Such dosing varies widely with the therapy: for example, capecitabine (2500 mg/m² orally) is dosed twice daily for 2 weeks on and 1 week off, imatinib mesylate (400 or 600 mg/day orally) is dosed daily, rituximab is dosed weekly, paclitaxel (135-175 mg/m²) and docetaxel (60-100 mg/m²) are dosed weekly to every three weeks, carboplatin (4-6 mg/mL·min) is dosed once every 3 or 4 weeks (though the doses may be split and administered over several days), nitrosourea alkylating agents such as carmustine are dosed as infrequently as once every 6 weeks. Radiotherapy may be administered as frequently as weekly (or even within that split into smaller dosages administered daily).

A person of ordinary skill in the art of cancer therapy will be able to ascertain a therapeutically effective amount of the GST-activated anticancer compound and a therapeutically effective amount of another anticancer therapy for a given cancer and stage of disease without undue experimentation and in reliance upon personal knowledge and the disclosure of this application.

The GST-activated anticancer compound and the another anticancer therapy may be administered by any route suitable to the subject being treated and the nature of the subject's condition. Routes of administration include, but are not limited to, administration by injection, including intravenous, intraperitoneal, intramuscular, and subcutaneous injection, by transmucosal or transdermal delivery, through topical applications, nasal spray, suppository and the like or may be administered orally. Formulations may optionally be liposomal formulations, emulsions, formulations designed to administer the drug across mucosal membranes or transdermal formulations. Suitable formulations for each of these methods of administration may be found, for example, in Remington: The Science and Practice of Pharmacy, 20th ed., A. Gennaro, ed., Lippincott Williams & Wilkins, Philadelphia, Pa., U.S.A. Typical formulations will be either oral (as for compounds such as capecitabine) or solutions for intravenous infusion. Typical dosage forms will be tablets (for oral administration), solutions for intravenous infusion, and lyophilized powders for reconstitution as solutions for intravenous infusion. Kits may contain the GST-activated anticancer compound as a dosage form, and the another chemotherapy agent, molecular targeted therapy agent, and/or biologic therapy agent, also in dosage form, for example packaged together in a common outer packaging.

Combinations considered of particular present interest are the combination administration of TLK286: with a platinum compound such as carboplatin or cisplatin, optionally in further combination with gemcitabine or a taxane such as docetaxel or paclitaxel; with gemcitabine; with a taxane; with an anthracycline such as doxorubicin or liposomal doxorubicin; with oxaliplatin, optionally in further combination with capecitabine or fluorouracil/leucovorin; and with gemcitabine or a platinum compound such as carboplatin or cisplatin, in further combination with a vinca alkaloid such as vinorelbine. It will be seen from the in vitro and therapeutic examples that follow that TLK286 is additive to synergistic with a variety of other cancer therapies, and, as mentioned previously, it is expected that TLK286 or other GST-activated anticancer compounds can be added to existing anticancer therapies generally.

In Vitro Examples

The following examples illustrate the beneficial effect of TLK286, a GST-activated anticancer compound, in combination with an other anticancer compound against human cancer cell lines in vitro. These results are considered predictive of efficacy in human cancer chemotherapy, as each of TLK286 and the other anticancer agent tested have shown anticancer activity in humans.

Cancer cell lines. The human cancer cell lines A549 (lung carcinoma), DLD-1 (colorectal adenocarcinoma), HT29 (colorectal adenocarcinoma), K-562 (chronic myelogenous leukemia), MCF-7 (breast adenocarcinoma), MG-63 (osteosarcoma), OVCAR-3 (ovarian adenocarcinoma), and RL (non-Hodgkin's B cell lymphoma) were obtained from the American Type Culture Collection, Manassas, Va., U.S.A. The human breast carcinoma cell line MN-1 was obtained from the National Cancer Institute, Bethesda, Md., U.S.A.

Anticancer compounds. Gefitinib and TLK286 were prepared by Telik. Carboplatin, cisplatin, doxorubicin, and paclitaxel were obtained from Sigma-Aldrich Chemical Company, St. Louis, Mo., U.S.A. Docetaxel was obtained from Aventis Pharmaceuticals Inc., gemcitabine from Eli Lilly and Company, oxaliplatin from Sanofi-Synthelabo Inc., and rituxan from IDEC Pharmaceuticals Corporation.

Assay methods. All assays were conducted in triplicate wells, with solvent control. The extent of cell growth was expressed as a percentage of the signal from the solvent control wells. The means were computed and graphed, with the standard deviations shown as error bars.

### EXAMPLE 1

### TLK286 Hydrochloride and Carboplatin

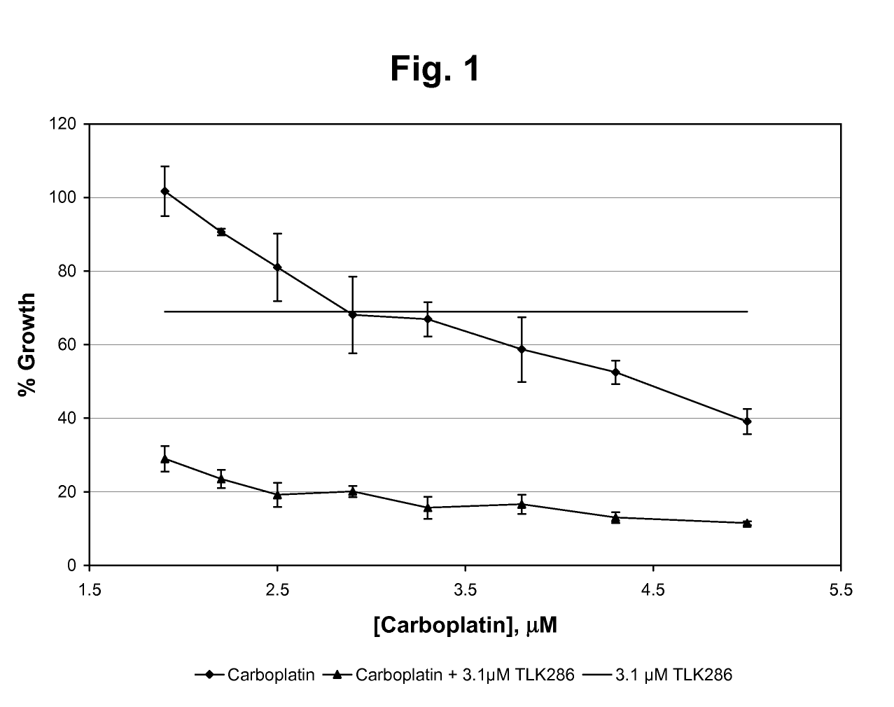

The human ovarian cancer cell line OVCAR-3 was seeded at 4×10⁴ cells/mL, 150 μL/well, and allowed to attach to the wells for 4-5 hours. The diluted compounds or solvent controls were then added at 50 μL/well. Incubation with TLK286 alone and in combination with carboplatin was continued for approximately three cell doublings, and cell viability was determined using the Wst-1 assay, where the plates were pulsed with the metabolic dye Wst-1 (Roche Diagnostics Corporation, Indianapolis, Ind., U.S.A.) (20 μL/well) and incubated for 1-2 hours. Each multiwell plate was read several times at 30 minute intervals to ensure linearity of detection. In various study designs using both fixed and variable ratios, there was a marked enhancement of cytotoxicity when TLK286 was combined with carboplatin compared to either compound alone. The results were further analyzed using the Combination Index (CI) method with the “CalcuSyn” program from Biosoft. A CI value of less than 1 indicates synergy, 1 indicates an additive effect, and greater than 1 indicates antagonism. This analysis indicated that combinations of TLK286 and carboplatin were generally synergistic with an average CI value of less than 1 in repeated experiments. FIG. 1 shows the activity of TLK286 (at 3.1 μM, about IC₃₀) and carboplatin (at concentrations between about 1.85 and 4 μM, from nearly no effect to nearly maximum inhibition), and clearly illustrates the beneficial effect of the combination.

### EXAMPLE 2

### TLK286 and Oxaliplatin

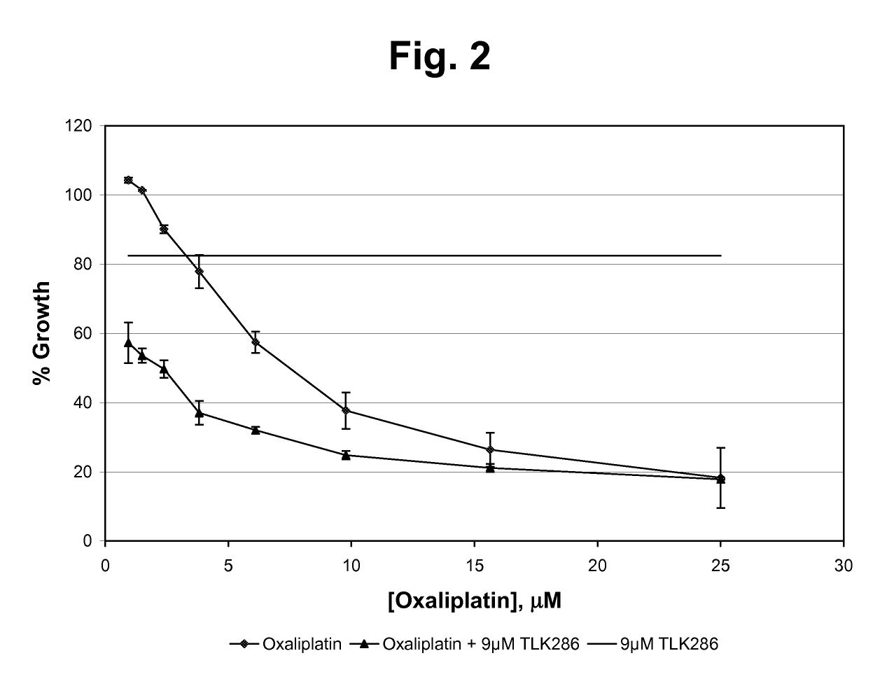

The human colon cancer cell line DLD-1 was seeded at 4×10⁴ cells/mL, 150 μL/well, and allowed to attach to the wells overnight. The diluted compounds or solvent controls were then added at 50 μL/well. Incubation with TLK286 alone and in combination with oxaliplatin was continued for approximately four cell doublings, and cell viability was determined using the CellTiter-Glo assay (Promega Corporation, Madison, Wis., U.S.A.), used in accordance with the assay kit directions. In various study designs, using both equal potency and variable ratios, there was a marked enhancement of cytotoxicity when TLK286 was combined with oxaliplatin compared to either compound alone. The results were further analyzed using the Combination Index (CI) method with the “CalcuSyn” program from Biosoft. A CI value of less than 1 indicates synergy, 1 indicates an additive effect, and greater than 1 indicates antagonism. This analysis indicated that combinations of TLK286 and oxaliplatin were generally synergistic with an average CI value of less than 1 in repeated experiments. FIG. 2 shows the activity of TLK286 (at 9 μM, about IC₂₀) and oxaliplatin (at concentrations between about 1 and 25 μM, from nearly no effect to nearly maximum inhibition), and clearly illustrates the beneficial effect of the combination. The synergistic growth inhibition of DLD-1 cells by TLK286 and oxaliplatin was seen independently of whether the drugs were applied simultaneously or sequentially (either TLK286 or oxaliplatin first), though the greatest synergistic effect was seen when TLK286 was applied before oxaliplatin. TLK286 and oxaliplatin were also assayed in the human colorectal cancer cell line HT-29, and a beneficial effect of the combination was also seen.

### EXAMPLE 3

### TLK286 and Doxorubicin

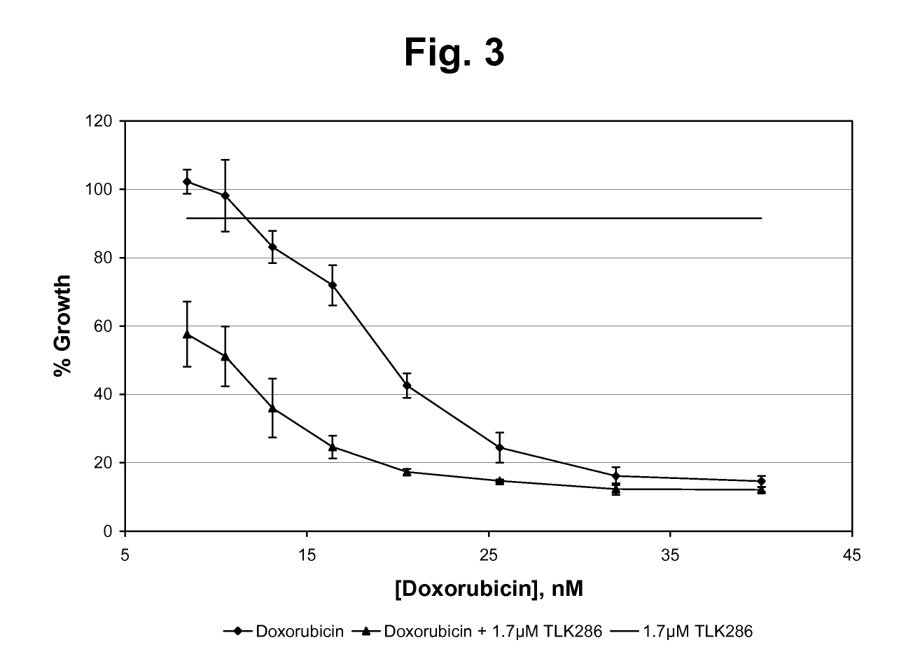

Doxorubicin is as a DNA intercalating agent that blocks DNA and RNA synthesis and affects topoisomerase II. Doxorubicin also alters membrane fluidity and generates semiquinone free radicals. The human chronic myelogenous leukemia cell line K-562, the human osteosarcoma cell line MG-63, and the human ovarian cancer cell line OVCAR-3 were each incubated with TLK286 alone and in combination with doxorubicin, and cell viability determined. The results were analyzed according to the Combination Index method with the “CalcuSyn” program from Biosoft. Synergy was observed when a concentration of doxorubicin between 10 and 20 nM was combined with a variable amount of TLK286. Data with all three cell lines showed that the combination of TLK286 and doxorubicin at fixed and variable ratios were synergistic to additive based on all the analyzable data points. FIG. 3 shows the activity of TLK286 (at 1.7 μM, about IC₁₀) and doxorubicin (at concentrations between about 8 and 40 nM, from nearly no effect to nearly maximum inhibition) in OVCAR-3 cells, and clearly illustrates the beneficial effect of the combination.

### EXAMPLE 4

### TLK286 and Docetaxel

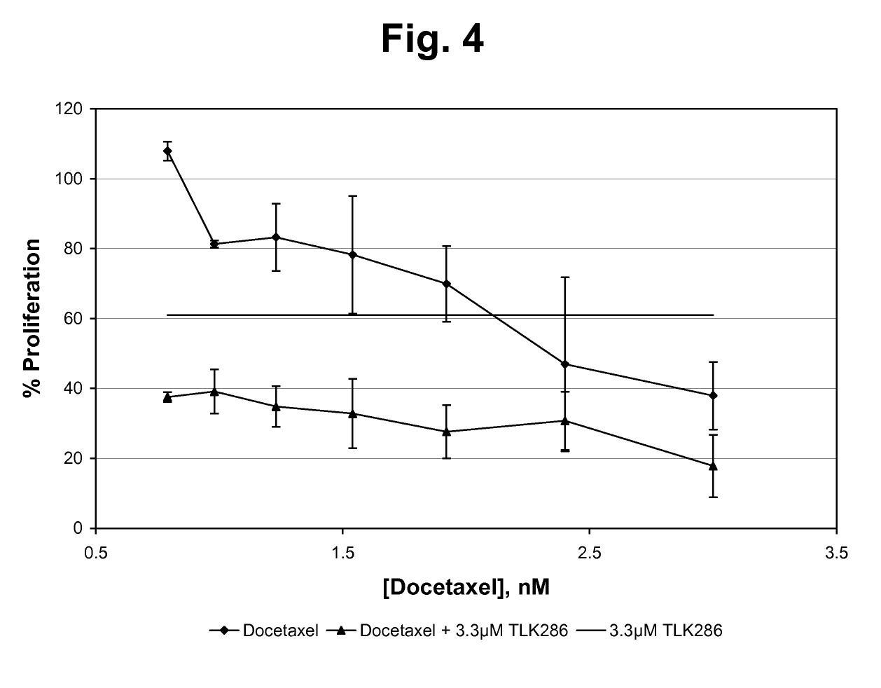

Since docetaxel is largely cytostatic for the human breast cancer cell line MCF-7, a cell proliferation assay was used. MCF-7 was seeded at 4×10⁴ cells/mL, 150 μL/well, and allowed to attach to the wells for 4-5 hours. The diluted compounds or solvent controls were then added at 50 μL/well. Incubation with TLK286 alone and in combination with docetaxel was continued for one doubling, and cell proliferation was determined using the BrdU (chemiluminescence) assay, by labeling with BrdU (Roche Diagnostics Corporation, Indianapolis, Ind., U.S.A.) overnight. The assay is based on the incorporation of BrdU, an analogue of thymidine, during DNA synthesis. The incorporation of BrdU, which reflects the extent of cell proliferation, was then quantitated with an ELISA kit (also from Roche Diagnostics Corporation). The results were analyzed according to the Combination Index method. Data using combinations of TLK286 and docetaxel at fixed and variable ratios were synergistic to additive. FIG. 4 shows the activity of TLK286 (at 3.3 μM, about IC₄₀) and docetaxel (at concentrations between about 0.8 and 3 nM, from nearly no effect to about ⁶⁰% inhibition) and, and clearly illustrates the beneficial effect of the combination.

### EXAMPLE 5

### TLK286 and Cisplatin

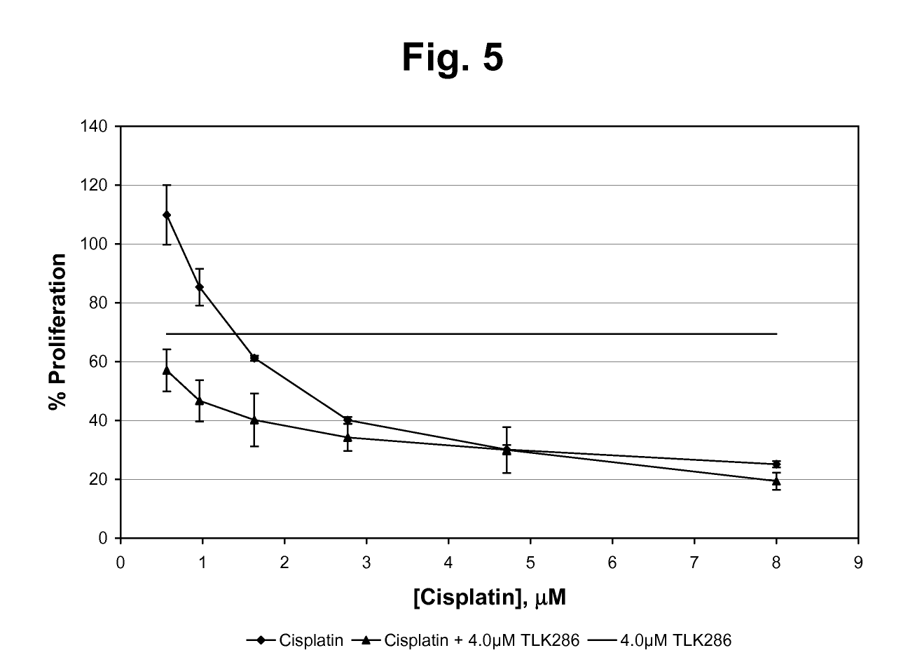

TLK286 and cisplatin were assayed in the human lung cancer cell line A-549, using a method similar to that of Example 4. FIG. 5 shows the activity of TLK286 (at4 μM, about IC₅₀) and cisplatin (at concentrations between about 0.5 and 8 μM, from nearly no effect to nearly maximum inhibition), and clearly illustrates the beneficial effect of the combination.

### EXAMPLE 6

### TLK286 and Paclitaxel

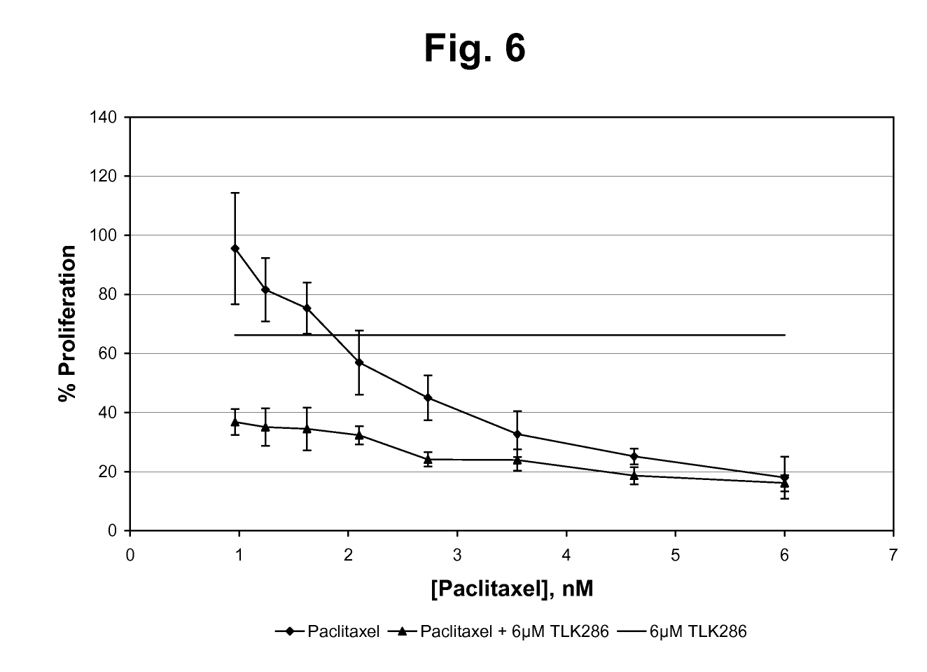

TLK286 and paclitaxel were assayed in the human lung cancer cell line A-549, using a method similar to that of Example 4. FIG. 6 shows the activity of TLK286 (at 6 μM and paclitaxel (at concentrations between about 1 and 6 nM, from nearly no effect to nearly maximum inhibition), and clearly illustrates the beneficial effect of the combination. TLK286 and paclitaxel were also assayed in the human ovarian cancer cell line OVCAR-3, and a beneficial effect of the combination was also seen.

### EXAMPLE 7

### TLK286 and Gemcitabine

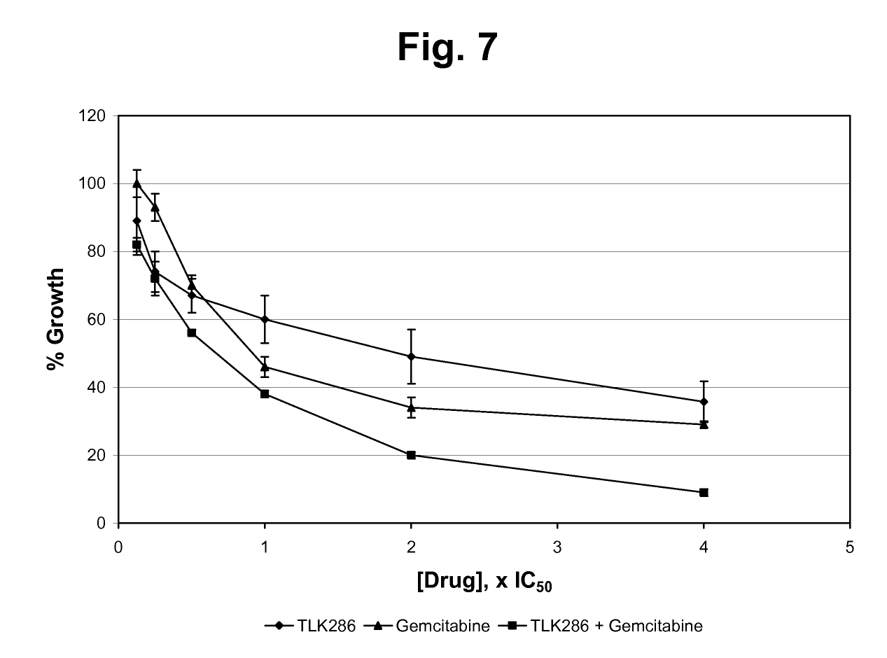

TLK286 and gemcitabine were assayed in the human breast cancer cell line MCF-7, using a method similar to that of Example 1. FIG. 7 shows the activity of TLK286 and gemcitabine, alone and in combination, at concentrations between about 0.1 and 4 IC₅₀, and clearly illustrates the beneficial effect of the combination.

### EXAMPLE 8

### TLK286 and Rituximab

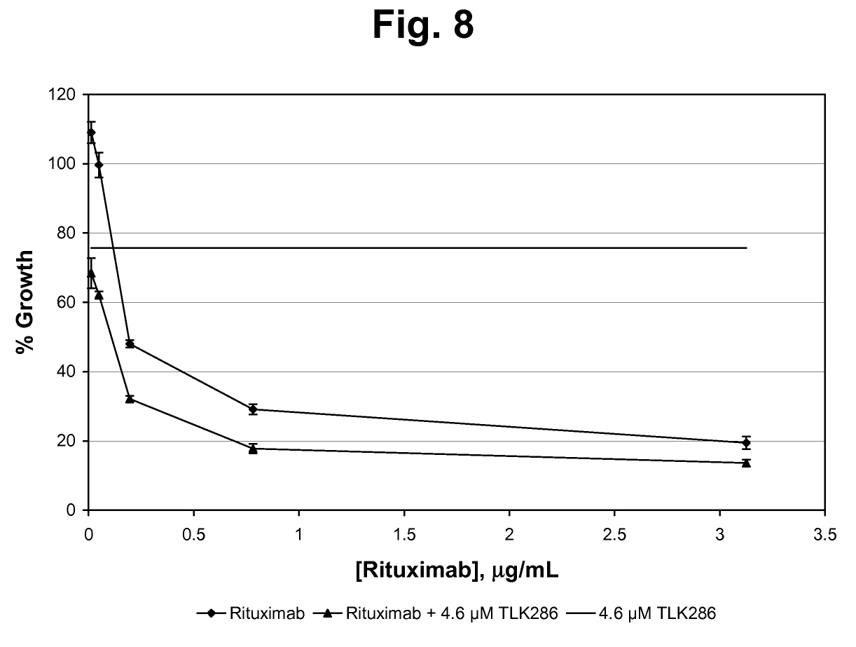

TLK286 and rituximab were assayed in the human non-Hodgkin's B cell lymphoma cell line RL, using a method similar to that of Example 2. FIG. 8 shows the activity of TLK286 (at 4.6 μM, about IC₂₅) and rituximab (at concentrations between about 0.01 and 3 μg/mL, from nearly no effect to nearly maximum inhibition), and clearly illustrates the beneficial effect of the combination.

### EXAMPLE 9

### TLK286 and Gefitinib

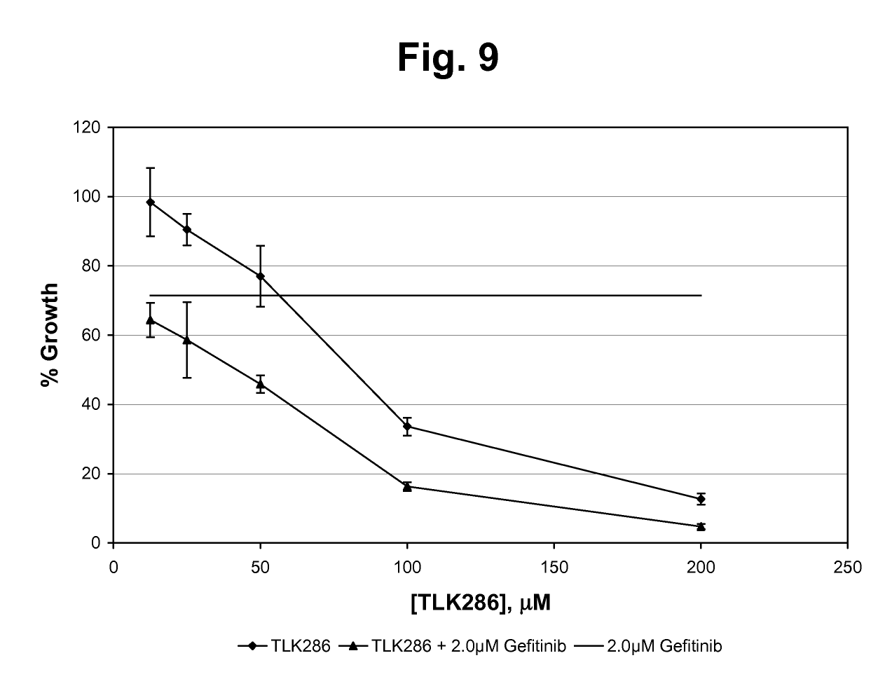

TLK286 and gefitinib were assayed in the human breast cancer cell line MX-1, using a method similar to that of Example 2. FIG. 9 shows the activity of TLK286 (at concentrations between about 12 and 200 μM, from nearly no effect to nearly maximum inhibition) and gefitinib (at 2.0 μM, about IC₃₀), and clearly illustrates the beneficial effect of the combination.

Therapeutic Examples

The following examples illustrate dosage regimens for TLK286, a GST-activated anticancer compound, in combination with another anticancer therapy.

### EXAMPLE 10

### Combination Therapy with TLK286 and Docetaxel in Non-Small Cell Lung Carcinoma

46 patients with Stage IIIB or Stage IV non-small cell lung carcinoma were enrolled in a clinical study, and 20 patients were evaluable for interim analysis. Of the 20 patients, all were resistant or refractory to platinum anticancer compounds, 16 were resistant or refractory to paclitaxel, and many had failed to respond to other chemotherapies, including gemcitabine, permetrexed, EFGR inhibitors such as erlotinib hydrochloride and gefitinib, and angiostatins. TLK286 at an initial dose of 500 mg/m² body surface area was administered intravenously, followed 30 minutes later by the intravenous administration of docetaxel at 75 mg/m². The TLK286 dose was increased to 750 mg/m² and further to 960 mg/m². Of the 20 patients, three have received TLK286 at 500 mg/m², three at 750 mg/m², and fourteen at 960 mg/m², in each case followed by 75 mg/m² docetaxel. Of the 14 patients at the 960 mg/m² TLK286 dose, 4 have shown a partial response, and 5 have shown stable disease, using RECIST (Response Evaluation Criteria in Solid Tumors) criteria; while all 3 patients at 750 mg/m² and 1 patient at 500 mg/m² TLK286 have shown stable disease. The study is ongoing, with administration of the drugs at 3-weekly intervals, and clearly illustrates the beneficial effect of the combination.

### EXAMPLE 11

### Combination Therapy with TLK286 and Carboplatin in Ovarian Carcinoma

13 patients with metastatic ovarian carcinoma were enrolled in a clinical study, and 8 patients were evaluable for interim analysis. Of the 8 patients, 6 were resistant or refractory to platinum anticancer compounds, all were resistant or refractory to paclitaxel, and many had failed to respond to other chemotherapies, including liposomal doxorubicin, gemcitabine, and topotecan. TLK286 at 500 mg/m² body surface area was administered intravenously, followed 30 minutes later by the intravenous administration of carboplatin at 5 or 6 mg/mL·min. Of the 8 patients, 1 has shown a complete response, 4 have shown a partial response, and 2 have shown stable disease. The study is ongoing, with administration of the drugs at 3- or 4-weekly intervals, including dose escalation with TLK286, and clearly illustrates the beneficial effect of the combination.

### EXAMPLE 12

### Combination Therapy with TLK286 and Liposomal Doxorubicin in Ovarian Carcinoma

17 patients with metastatic ovarian carcinoma were enrolled in a clinical study, and 13 patients were evaluable for interim analysis. Of the 13 patients, all were resistant or refractory to platinum anticancer compounds, 9 were resistant or refractory to paclitaxel, and many had failed to respond to other chemotherapies (the median number of prior chemotherapeutic regimens was two). TLK286 at an initial dose of 500 mg/m² body surface area was administered intravenously, followed 30 minutes later by the intravenous administration of liposomal doxorubicin at 40 mg/m². The TLK286 dose was increased to 750 mg/m² and further to 960 mg/m², and the liposomal doxorubicin dose was increased to 50 mg/m². Of the 17 patients, 3 have received TLK286 at 500 mg/m², 3 at 750 mg/m², and 4 at 960 mg/m², in each case followed by 40 mg/m² liposomal doxorubicin, and 7 patients have received TLK286 at 960 mg/m² followed by 50 mg/m² liposomal doxorubicin. Of the 3 evaluable patients at the 960 mg/m² TLK286/50 mg/m² liposomal doxorubicin dose, 1 has shown a partial response and 1 has shown stable disease; while 2 of 3 evaluable patients at 960 mg/m² TLK286/40 mg/m² liposomal doxorubicin, 1 of 3 at 750 mg/m²/40 mg/m², and 1 of 3 at 500 mg/m²/40 mg/m² have shown stable disease. The study is ongoing, with administration of the drugs at 4-weekly intervals, and clearly illustrates the beneficial effect of the combination.

Combination Therapy with TLK286 and Other Anticancer Therapies

TLK286 at an initial dose of 500 mg/m² is administered intravenously, followed 30 minutes later by the intravenous administration of oxaliplatin at a therapeutically effective dose such as 85 mg/m². The TLK286 dose may be increased to 850 mg/m² and further to 1280 mg/m², and the oxaliplatin dose may also be varied. This combination is administered at 2-weekly intervals.

TLK286 at an initial dose of 500 mg/m² is administered intravenously at 3-weekly intervals, accompanied by the oral administration of capecitabine at a therapeutically effective dose such as 1250 mg/m² twice/day for 14 days, followed by 7 days without treatment. The TLK286 dose may be increased to 750 mg/m² and further to 960 mg/m², and the capecitabine dose may also be varied.

TLK286 at an initial dose of 400 mg/m² is administered intravenously at 2-weekly intervals, followed 30 minutes later by the intravenous administration of fluorouracil at a therapeutically effective dose such as 12 mg/Kg, with leucovorin rescue after completion of four days of fluorouracil therapy. The TLK286 dose may be increased to 700 mg/m² and further to 1000 mg/m², and the fluorouracil dose may also be varied.

Other GST-activated anticancer compounds may be used similarly in the method of this invention. Different other anticancer therapies, such as other chemotherapies, molecularly targeted therapies, biologic therapies, and radiation therapies may also be used similarly in the method of this invention;

While this invention has been described in conjunction with specific embodiments and examples, it will be apparent to a person of ordinary skill in the art, having regard to that skill and this disclosure, that equivalents of the specifically disclosed materials and methods will also be applicable to this invention; and such equivalents are intended to be included within the following claims. cm We claim:

1. A method of combination cancer therapy in a human comprising administering a therapeutically effective amount of a compound that is canfosfamide or a salt thereof and a therapeutically effective amount of another anticancer therapy.

2. The method of claim 1 where the compound is canfosfamide hydrochloride.

3. The method of claim 1 where the another anticancer therapy is selected from one or more of chemotherapy, molecular targeted therapy, biologic therapy, and radiotherapy.

4. The method of claim 3 where the another anticancer therapy is administration of one or more of an alkylating agent, an antimetabolite, a natural product, a hormone or hormone antagonist, a miscellaneous agent, a functional therapeutic agent, a gene therapy agent, an antisense therapy agent, a tyrosine kinase inhibitor, a gene expression modulator, a phenotype-directed therapy agent, a monoclonal antibody, an immunotoxin, a radioimmunoconjugate, a cancer vaccine, an interferon, and an interleukin.

5. The method of claim 4 where the another anticancer therapy is administration of one or more of busulfan, thiotepa, chlorambucil, cyclophosphamide, estramustine, ifosfamide, mechlorethamine, melphalan, uramustine, carmustine, lomustine, streptozocin, dacarbazine, procarbazine, temozolamide, cisplatin, carboplatin, oxaliplatin, satraplatin, (SP-4-3)-(cis)-amminedichtoro-[2-methylpyridine]-platinum(II), methotrexate, permetrexed, raltitrexed, trimetrexate, cladribine, chlorodeoxyadenosine, clofarabine, fludarabine, mercaptopurine, pentostatin, thioguanine, azacitidine, capecitabine, cytarabine, edatrexate, floxuridine, fluorouracil, gemcitabine, troxacitabine, bleomycin, dactinomycin, mithramycin, mitomycin, mitoxantrone, porfiromycin, daunorubicin, daunorubicin, doxorubicin, liposomal doxorubicin, epirubicin, idarubicin, valrubicin, L-asparaginase, PEG-L-asparaginase, paclitaxel, docetaxel, vinblastine, vincristine, vindesine, vinorelbine, irinotecan, topotecan, amsacrine, etoposide, teniposide, fluoxymesterone, testolactone, bicalutamide, cyproterone, flutamide, nilutamide, aminoglutethimide, anastrozole, exemestane, formestane, letrozole, dexamethasone, prednisone, diethylstilbestrol, fulvestrant, raloxifene, tamoxifen, toremifine, buserelin, goserelin, leuprolide, triptorelin, medroxyprogesterone acetate, megestrol acetate, levothyroxine, liothyronine, altretamine, arsenic trioxide, gallium nitrate, hydroxyurea, levamisole, mitotane, octreotide, procarbazine, suramin, thalidomide, methoxsalen, sodium porfimer, bortezomib, erlotinib hydrochloride, gefitinib, imatinib mesylate, semaxanib, adapalene, bexarotene, trans-retinoic acid, 9-cis-retinoic acid, and N-(4-hydroxyphenyl)retinamide, alemtuzumab, bevacizumab, cetuximab, ibritumomab tiuxetan, rituximab, trastuzumab, gemtuzumab ozogamicin, ¹³¹I-tositumomab, interferon-α₂ₐ, interferon-α_{2b}, aldesleukin, denileukin diftitox, and oprelvekin.

6. The method of claim 5 where the another anticancer therapy is administration of: a platinum compound, optionally in further combination with gemcitabine or a taxane; gemcitabine; a taxane; an anthracycline; oxaliplatin, optionally in further combination with capecitabine or fluorouracil/leucovorin; and gemcitabine or a platinum compound, in further combination with a vinca alkaloid.

7. The method of claim 3 where the another anticancer therapy is administration of two or more of chemotherapy; molecular targeted therapy; biologic therapy; and radiotherapy.

8. The method of claim 7 where the another anticancer therapy is administration of two or more chemotherapy agents.

9. The method of claim 1 where the dosing of the compound is about 60-1280 mg/m² body surface area at 1-35 day intervals.

10. The method of claim 9 where the dosing is about 500-1000 mg/m² at 1-5 week intervals.

11. The method of claim 10 where the compound is canfosfamide hydrochloride and the dosing is about 500-1000 mg/m² at 1, 2, 3, or 4 week intervals.

12. A method of potentiating the effect of an anticancer therapy in a human, comprising administering a therapeutically effective amount of a compound that is canfosfamide or a salt thereof to the human being treated with the anticancer therapy.

13. The method of claim 12 where the compound is canfosfamide hydrochloride.

14. The method of claim 12 where the another anticancer therapy is selected from one or more of chemotherapy, molecular targeted therapy, biologic therapy, and radiotherapy.

15. The method of claim 14 where the another anticancer therapy is administration of one or more of an alkylating agent, an antimetabolite, a natural product, a hormone or hormone antagonist, a miscellaneous agent, a functional therapeutic agent, a gene therapy agent, an antisense therapy agent, a tyrosine kinase inhibitor, a gene expression modulator, a phenotype-directed therapy agent, a monoclonal antibody, an immunotoxin, a radioimmunoconjugate, a cancer vaccine, an interferon, and an interleukin.

16. The method of claim 15 where the another anticancer therapy is administration of one or more of busulfan, thiotepa, chlorambucil, cyclophosphamide, estramustine, ifosfamide, mechlorethamine, melphalan, uramustine, carmustine, lomustine, streptozocin, dacarbazine, procarbazine, temozolamide, cisplatin, carboplatin, oxaliplatin, satraplatin, (SP-4-3)-(cis)-amminedichtoro-[2-methylpyridine]-platinum(II), methotrexate, permetrexed, raltitrexed, trimetrexate, cladribine, chlorodeoxyadenosine, clofarabine, fludarabine, mercaptopurine, pentostatin, thioguanine, azacitidine, capecitabine, cytarabine, edatrexate, floxuridine, fluorouracil, gemcitabine, troxacitabine, bleomycin, dactinomycin, mithramycin, mitomycin, mitoxantrone, porfiromycin, daunorubicin, daunorubicin, doxorubicin, liposomal doxorubicin, epirubicin, idarubicin, valrubicin, L-asparaginase, PEG-L-asparaginase, paclitaxel, docetaxel, vinblastine, vincristine, vindesine, vinorelbine, irinotecan, topotecan, amsacrine, etoposide, teniposide, fluoxymesterone, testolactone, bicalutamide, cyproterone, flutamide, nilutamide, aminoglutethimide, anastrozole, exemestane, formestane, letrozole, dexamethasone, prednisone, diethylstilbestrol, fulvestrant, raloxifene, tamoxifen, toremifine, buserelin, goserelin, leuprolide, triptorelin, medroxyprogesterone acetate, megestrol acetate, levothyroxine, liothyronine, altretamine, arsenic trioxide, gallium nitrate, hydroxyurea, levamisole, mitotane, octreotide, procarbazine, suramin, thalidomide, methoxsalen, sodium porfimer, bortezomib, erlotinib hydrochloride, gefitinib, imatinib mesylate, semaxanib, adapalene, bexarotene, trans-retinoic acid, 9-cis-retinoic acid, and N-(4-hydroxyphenyl)retinamide, alemtuzumab, bevacizumab, cetuximab, ibritumomab tiuxetan, rituximab, trastuzumab, gemtuzumab ozogamicin, ¹³¹I-tositumomab, interferon-α₂ₐ, interferon-α_{2b}, aldesleukin, denileukin diftitox, and oprelvekin.

17. The method of claim 16 where the another anticancer therapy is administration of: a platinum compound, optionally in further combination with gemcitabine or a taxane; gemcitabine; a taxane; an anthracycline; oxaliplatin, optionally in further combination with capecitabine or fluorouracil/leucovorin; and gemcitabine or a platinum compound, in further combination with a vinca alkaloid.

18. The method of claim 14 where the another anticancer therapy is administration of two or more of chemotherapy; molecular targeted therapy; biologic therapy; and radiotherapy.

19. The method of claim 18 where the another anticancer therapy is administration of two or more chemotherapy agents.

20. The method of claim 12 where the dosing of the compound is about 60-1280 mg/m² body surface area at 1-35 day intervals.

21. The method of claim 20 where the dosing is about 500-1000 mg/m² at 1-5 week intervals.

22. The method of claim 21 where the compound is canfosfamide hydrochloride and the dosing is about 500-1000 mg/m² at 1, 2, 3, or 4 week intervals.

23. A pharmaceutical composition for anticancer therapy comprising:
a compound that is canfosfamide or a salt thereof,
one or more of another anticancer chemotherapy agent, a molecular targeted therapy agent, and a
biologic therapy agent, and
an excipient.

24. The composition of claim 23 where the compound is canfosfamide hydrochloride.
